(19) United States
(12) Patent Application Publication
(10) Pub. No.: US 2024/0293591 A1
(43) Pub. Date: Sep. 5, 2024

(54) ULTRAVIOLET DISINFECTING APPARATUS

(71) Applicant: Finsen Technologies Limited, London, Greater London (GB)

(72) Inventor: Tristan Willaims, London, Greater London (GB)

(21) Appl. No.: 18/694,198

(22) PCT Filed: Sep. 23, 2022
(86) PCT No.: PCT/GB2022/052407

§ 371 Date: Mar. 21, 2024

(30) Foreign Application Priority Data
Sep. 24, 2021 (GB) 2113650.2

(51) Int. Cl. A61L 2/10; A61L 2/24; A61L 9/20

(52) U.S. Cl. CPC A61L 2/10; A61L 2/24; A61L 9/20; A61L 2202/11; A61L 2202/25

(57) ABSTRACT

There is provided an ultraviolet disinfecting apparatus for disinfecting a room, the apparatus comprising: a support structure with a base region; a first set of UV emitters supported by the support structure to form a perimeter about which UV light can be emitted into said room; and a second set of UV emitters supported by the support structure such that they can emit UV light into a region of said room which lies beneath the base region and inside the perimeter.

### FIELD OF THE INVENTION

The present invention concerns an ultraviolet (UV) disinfecting apparatus. More particularly, but not exclusively, this invention concerns ultraviolet disinfecting apparatus for disinfecting a room. The invention also concerns a room steriliser and floor-standing disinfecting apparatus for disinfecting a room.

### BACKGROUND OF THE INVENTION

Use of ultraviolet (UV) light for disinfection of medical equipment is known in the art. UV-C is high frequency UV radiation, which tests have shown kills all known pathogens at a wavelength between 250 and 260 nm. UV-C is suitable for air, water, and surface disinfection. Apparatus for disinfecting a room using UV radiation are known.

Such apparatus sometimes comprise a set of UV-C tubes aligned vertically and positioned around a central pillar. In use, the tubes are energised to emit UV-C light into the room so as to disinfect it. An example of one such apparatus is shown in WO2017216533.

In such apparatus, a ‘shadow’ in the UV illumination can be created, for instance beneath the apparatus. This shadow can receive lower doses of UV light than would be preferred. This can mean that the tubes may need to be energised for longer periods than would otherwise be necessary, so as to ensure that the area in the shadow receives a sufficient dose of UV light, or can mean that an additional disinfection step (such as moving and restarting the apparatus, or using a chemical disinfectant) is required. This, in turn, can increase the overall time needed to adequately disinfect a room.

The present invention seeks to mitigate the above-mentioned problem. Alternatively or additionally, the present invention seeks to provide an improved or alternative ultraviolet disinfecting apparatus, room steriliser or floor-standing disinfecting apparatus.

### SUMMARY OF THE INVENTION

According to a first aspect of the present invention there is provided ultraviolet disinfecting apparatus for disinfecting a room, the apparatus comprising:
a support structure with a base region;
a first set of UV emitters supported by the support structure to form a perimeter about which UV light can be emitted into said room; and
a second set of UV emitters supported by the support structure such that they can emit UV light into a region of said room which lies beneath the base region and inside the perimeter.

The two sets of UV emitters can improve the speed with which a room can be disinfected by UV light. For example in the case where the apparatus is placed in a room with its base region on the floor of the room, the first set of UV emitters may sterilise the majority of the room, but a ‘shadow’ of reduced illumination may be formed beneath the base region and inside the perimeter. The second set of UV emitters emitting UV light into that area can reduce or eliminate this shadow, thereby reducing the need for the apparatus to be left energised solely to provide a sufficient dose of UV light to that area and/or the need for a second disinfecting step to be performed.

For the avoidance of doubt, a ‘set’ of UV emitters may be a single emitter. In the case of the first set of emitters being a single emitter, the perimeter may be formed by the shape of the single emitter (for instance if that emitter is a fluorescent tube following an arcuate or helical path) or by a path of movement of the emitter (for example the perimeter may be formed by an orbital path followed by the emitter).

The first and/or second set of UV emitters may be a plurality of emitters, for instance an array of emitters. For example, the first set of emitters may be an arcuate or generally annular array of emitters extending around the perimeter, and/or the second set of emitters may be a generally planar array which may be oriented generally horizontally. A set comprising plurality of emitters may advantageously increase the coverage of UV light which can be provided by that set, and thus by the apparatus as a whole. Instead or as well, a set comprising a plurality of emitters may produce a greater overall UV output than may be possible with a single emitter.

The UV emitters of the first set may each take the form of a mercury vapour lamp. This may allow the first set of emitters to operate at a beneficially high level of energy efficiency, providing advantageously powerful UV radiation for a given electrical power, thereby allowing the apparatus as a whole to be advantageously energy efficient.

The UV emitters of the second set may each take the form of an LED lamp. This may allow the second set to be advantageously compact.

In some embodiments, the UV emitters of the first set each take the form of a mercury vapour lamp, and the UV emitters of the second set each take the form of an LED lamp.

This can allow the apparatus to utilise different types of UV emitter for different circumstances, according to the benefits provided by that type of emitter. This is discussed in more detail below.

According to a second aspect of the present invention there is provided ultraviolet disinfecting apparatus for disinfecting a room, the apparatus comprising a support structure which supports a first set of UV emitters each taking the form of a mercury vapour lamp, and a second set of UV emitters each taking the form of an LED lamp.

As sources of UV light, mercury vapour lamps and LED vapour lamps offer different advantages which make them more suited to different circumstances. For example, as discussed above mercury vapour lamps may be advantageously energy efficient and LEDs may be advantageously compact. Utilising both mercury vapour lamps and LED lamps may allow the apparatus to utilise UV emitters the characteristics of which are tailored to the circumstances in which they are to be used. For example, in an apparatus where the first set of UV emitters is used to irradiate and disinfect a room as a whole, the energy efficiency of mercury vapour lamps can be of particular benefit. Conversely, if in that apparatus the second set of UV emitters is positioned on the underside of the apparatus and used to irradiate and disinfect the space beneath it, the compactness provided by LEDs can be of particular benefit since there may be less space available (particularly if the apparatus is wheeled and needs to have a relatively high ground clearance).

In the second aspect of the invention, the support structure may support the first and second sets of UV emitters such that they can emit UV light in different directions.

The first and second sets emitting light in different directions can allow each set to emit light towards a location suited to its type of lamp. For instance in the above example where the first set is used to irradiate a room as a whole and the second set is used to irradiate the floor beneath the apparatus where there would otherwise be a shadow, the two sets emitting light in different directions can allow the apparatus to be positioned for both functions to take place—the first set can face into the room as a whole and the second set can face the floor.

The direction in which a set of emitters emits light may be considered to be an average direction in which light is emitted (for instance if an emitter emits light as a divergent cone the direction may be considered to be along the axis of that cone), or may be considered to be a direction in which the brightest light is emitted.

In the second aspect of the invention, the support structure may comprise a base region, supports the first set of UV emitters such that they can emit UV light into said room (for example towards the walls of the room), and supports the second set of UV emitters such that they can emit UV light into a region of the room which lies beneath the base region.

This can allow the mercury vapour lamps to disinfect the room as a whole and the LEDs to disinfect the floor beneath the apparatus, as discussed above, with advantageous ease.

As an alternative, the support structure may support the first set of UV emitters such that they can emit UV light into said room, and support the second set of UV emitters such that they can emit light into a different region of the room (which may, in use, correspond to an area of ceiling, a corner of a room or a location under a piece of furniture).

In the second aspect of the invention, the first set of UV emitters may be supported by the support structure to form a perimeter about which UV light can be emitted into said room, and the second set of UV emitters may be supported by the support structure such that they can emit UV light into a region of the room which lies beneath the base region and inside the perimeter.

The first set of UV emitters forming the perimeter may allow the apparatus to irradiate a large portion of a room at once, for example if the first set of UV emitters extend all around the perimeter of the apparatus, then the apparatus could irradiate all around itself at once. However, the space within the perimeter (for example within the perimeter but below the first set of UV emitters) may be particularly susceptible to not receiving sufficient radiation. The second set of UB emitters emitting UV light into this region can therefore be of particular benefit.

As one alternative, the second set of UV emitters may be supported by the support structure such that they can emit UV light into a region of the room which lies beneath the base region but outside the perimeter. This may still provide a benefit in terms of reducing the shadow beneath the apparatus, even if the floor beneath the apparatus and inside the perimeter does not get irradiated.

In either aspect of the invention, the first set of UV emitters may extend all the way around the perimeter.

This can allow the first set of UV emitters to emit UV light for 360 degrees around the apparatus, thereby providing advantageously high room coverage.

It is to be understood that a set of UV emitters may be considered to extend all the way around the perimeter if the UV light therefrom is emitted through 360 degrees around the perimeter. For the avoidance of doubt, there may be one or more circumferential gaps between adjacent emitters in a set which extends all the way around the perimeter.

As an alternative, the first set of emitters may extend through only part of the perimeter, for example at least 180 degrees or at least 270 degrees around the perimeter.

In either aspect of the invention, the second set of UV emitters may be positioned inside the perimeter.

This may provide a particularly simple layout in which the UV emitters of the second set can emit UV light onto a region of the room which lies inside the perimeter. Instead or as well, this location for the second set of UV emitters may be beneficial in terms of shielding them from accidental knocks and/or avoiding them obscuring the first set of UV emitters.

As one alternative, the second set of UV emitters may be positioned outside of the perimeter. Such an arrangement may nonetheless allow the second set of UV emitters to irradiate the region of the room which lies beneath the base region and inside the perimeter. For example, the emitters may be provided outside of the perimeter but angled to direct their radiation inwards.

In either aspect of the invention, the second set of UV emitters may be positioned on the base region of the support structure.

This may provide a particularly simple layout in which the UV emitters of the second set can emit UV light onto a region of the room which lies beneath the base region. Instead or as well, this location for the second set of UV emitters may be beneficial in terms of shielding them from accidental knocks and/or avoiding them obscuring the first set of UV emitters.

As one alternative, the second set of UV emitters may be positioned elsewhere on the apparatus, for instance above the base region but positioned so as to emit UV light beneath it.

In either aspect of the invention:
each of said LED lamps may be provided on a thermally conductive backing;
the support structure may comprise a thermally conductive structural component; and
the thermally conductive backing and the thermally conductive structural component may be in thermal contact such that heat generated by the LED lamp is conducted into the thermally conductive structural component.

In such an arrangement all of the LED lamps may be provided on a single common thermally conductive backing, or there may be multiple thermally conductive backings. Where multiple thermally conductive backings are provided, they may each be in thermal contact with a common thermally conductive structural component, or different thermally conductive backings may be in thermal contact with different thermally conductive structural components.

The/each thermally conductive backing being in thermal contact with a thermally conductive structural component can provide advantageously efficient cooling of the LED lamps, as heat can be spread to (and dissipated from) a larger volume. In the absence of this thermal contact, heat may build up in the thermally conductive backing(s). This, in turn, could require the LED lamps to be run at lower power and/or require a relatively complex, bulky, or expensive cooling system to be provided.

The term ‘thermal contact’ should be interpreted to mean significant direct contact such that heat can pass therebetween or contact via a thermally conductive medium such as a metal adaptor plate or a layer of thermal paste. The thermally conductive structural component preferably functions as a heat sink. For example, the heat is preferably dissipated into the surrounding environment in a manner that does not adversely impact heat sensitive components in the apparatus.

The term ‘thermally conductive’ is intended to refer to a component made of a material (or materials) with a thermal conductivity of at least 1 W/mK, for instance at least 2 W/mK, at least 5 W/mK or at least 10 W/mK.

The thermally conductive backing and/or the thermally conductive structural component may be very thermally conductive, for instance being made of a material (or materials) with a thermal conductivity of at least at least 50 W/mK, at least 100 W/mK or at least 200 W/mK.

The thermally conductive backing may be a metal backing. Instead or as well, the thermally conductive structural component may be a metal structural component.

Each of said LED lamps is electrically connected to an LED driver circuit which is spaced apart from said LED lamp.

In such an arrangement each LED lamp may be electrically connected to (and spaced apart from) a common LED driver circuit, or each LED lamp may have an individual LED driver circuit connected thereto (and spaced apart therefrom).

The LED lamps being spaced apart from their driver circuit(s) can minimise number of components, and thus the space taken up, immediately adjacent to the LED lamps. This can, for example, avoid the driver circuit(s) obstructing the LED lamps, allow the LED lamps to be positioned closer together, and/or provide a structure which is more resilient to accidental knocks. As another example, where the LED lamps are provided on the base region of an apparatus provided with wheels, the driver circuit(s) being spaced apart therefrom can avoid the driver circuit(s) projecting towards the floor and thereby reducing the ground clearance of the apparatus (which may need to be relatively high in order for the apparatus to be wheeled over door thresholds and the like).

The first set of UV emitters may take the form of an array of generally vertically-oriented fluorescent tube lamps.

This may provide an arrangement which is particularly quick or efficient at disinfecting the space inside rooms.

As an alternative, the first set of UV emitters may take the form of another type of lamp, such as a mercury vapour spotlight.

Optionally:
the apparatus may comprise a generally upright column;
the first set of UV emitters may be supported by the support structure so that in use they emit UV light generally radially outwards from said column; and
the second set of UV emitters may be supported by the support structure so that in use they emit UV light generally axially downwards from said column.

This may provide an arrangement which is particularly well suited to whole-room disinfection, with the first set of UV emitters shining outwards from the column into the room as a whole, and the second set of UV emitters shining down to reduce or eliminate a shadow which would otherwise be present beneath the column.

As one alternative, the first and/or second sets of UV emitters may be supported so that in use they emit UV light in any other suitable direction. For example, the first set of UV emitters may be supported by the support structure so that in use they emit UV light generally axially upwards, and the second set of UV emitters may be supported by the support structure so that in use they emit UV light generally axially downwards. As another example the first set of UV emitters may be supported by the support structure so that in use, they emit UV light generally radially outwards, but the second set of UV emitters may be supported by the support structure so that in use they emit UV light generally axially upwards.

The first set of UV emitters may comprise two sub-sets of UV emitters which are movable relative to one another.

This may allow the first set of UV emitters to irradiate the desired part of a room advantageously thoroughly, for instance minimising the existence of shadows or dim areas, and/or may allow for easier storage. For example, the first set of UV emitters may comprise a fixed subset positioned relatively low down and a second subset which is movable generally upwards away from the base region, so that the first subset can irradiate the lower portion of a room and the second subset can be raised to irradiate the upper portion of the room. The second subset being movable would allow their height to be adjusted so as to be appropriate for the height of the room in question, and/or minimised for storage or transport.

The first and second sets of UV emitters may be in communication with a controller, the controller being configured to illuminate both sets at the same time.

The apparatus may comprise a safety shut-off system which may comprise, for example, an emergency stop button or a movement sensor. The emergency shut-off system may be arranged to selectively deactivate both sets of UV emitters.

Said UV emitters may be configured to emit light in the UVC frequency band. More particularly, said UV emitters may be configured to emit light with a wavelength of at least 240 nm or at least 250 nm, but no more than 280 nm or no more than 270 nm.

The apparatus may have a height of at least 25 cm, for instance at least 50 cm, at least 75 cm or at least 1 m. Instead or as well, the apparatus may have a width of at least 20 cm, for instance at least 30 cm or at least 40 cm. Instead or as well, the apparatus may have a width of at least 20 cm, for instance at least 30 cm or at least 40 cm. Instead or as well, the apparatus may have a horizontal length of at least 20 cm, for instance at least 30 cm or at least 40 cm.

Such a relatively large apparatus may allow more UV emitters to be provided thereupon, thereby increasing disinfection performance. Instead or as well, a relatively large apparatus can provide more space for other components such as batteries (where the apparatus is battery powered), sensing systems, cooling systems, control systems and the like. Furthermore, a relatively large apparatus may allow the UV emitters to be spread over a wider space, thereby reducing the effect of shadow-casting obstructions such as furniture.

The apparatus may have a height of no more than 2.5 m, for instance no more than 2 m or no more than 1.5 m. Instead or as well, the apparatus may have a width of no more than 1.5 m, for instance no more than 1 m or no more than 75 cm. Instead or as well, the apparatus may have a horizontal length of no more than 1.5 m, for instance no more than 1 m or no more than 75 cm.

Notwithstanding the aforementioned advantages of a relatively large apparatus, the apparatus staying within dimensions such as the above can enable it to be moved from room to room more easily, if required. For instance it may have a height small enough and a width and/or length small enough to fit through a conventional double door frame or single door frame.

The apparatus may be approximately man-sized. Most rooms, and other such spaces such as corridors, inbuilt cupboards, ambulances, and the like have a size and layout such that a person can stand in them. The apparatus being sized accordingly may allow it to be placed in, or moved through, such places with advantageous case.

According to a third aspect of the present invention there is provided a room steriliser comprising a first ultraviolet illuminator arranged to emit UV light in a generally outward direction during use, and a second ultraviolet illuminator arranged to emit UV light in a generally downward direction during use.

With the first illuminator emitting light outwards the majority of a room may be irradiated and thus sterilised with advantageous ease, but a shadow may be present beneath the steriliser. The second illuminator emitting light generally downwards can reduce or eliminate that shadow, thereby increasing the sterilisation performance of the steriliser as a whole. Otherwise, it may be necessary to disinfect the space beneath the steriliser in a separate step, or to irradiate the room at greater intensity or for a longer time than would otherwise be necessary.

According to a fourth aspect of the present invention there is provided ultraviolet disinfecting apparatus for disinfecting a room, the apparatus comprising a primary set of UV emitters arranged to emit UV light towards the walls of said room, and a secondary set of UV emitters arranged to emit UV light towards the floor of said room.

With the primary set of UV emitters emitting light towards the walls of the room, the majority of a room may be irradiated and thus disinfected with advantageous ease, but a shadow may be present on the floor of the room, particularly under the apparatus. The secondary set of UV emitters emitting light towards the floor of the room can reduce or eliminate any such shadow, thereby increasing the amount of the room which can be disinfected by the apparatus while in a particular position within the room, in a particular time. The second set are preferably located below, and inwards of, the primary set of emitters.

According to a fifth aspect of the present invention there is provided floor-standing disinfecting apparatus for disinfecting a room, the apparatus comprising an upright section with an array of generally vertical UV-emitting tubes, and an array of UV-emitting LEDs positioned on a lower end face of the upright section.

The generally vertical UV-emitting tubes can be particularly effective at irradiating, and thus disinfecting, the majority of a room. There may, however, be a shadow of reduced irradiation beneath the upright section. The UV-emitting LEDs on the lower end face of the upright section can reduce or eliminate that shadow.

According to a sixth aspect of the present invention there is provided an ultraviolet disinfecting apparatus for disinfecting a room, the apparatus comprising: a base having an upper side and a lower side; a first plurality of UV emitters upstanding from the upper side of the base and configured to emit UV light into the room; and a second plurality of UV emitters comprising an array of LED lamps arranged on the lower side of the base and configured to emit light into a region beneath the ultraviolet disinfecting apparatus during use.

It will of course be appreciated that features described in relation to one aspect of the present invention may be incorporated into other aspects of the present invention, where appropriate.

### DESCRIPTION OF THE DRAWINGS

Embodiments of the present invention will now be described by way of example only with reference to the accompanying schematic drawings in which:

FIG. 1 is a front view of an ultraviolet disinfecting apparatus according to an embodiment of the invention, in a retracted configuration;

FIG. 2 is a front view of the apparatus of FIG. 1, in an extended configuration;

FIG. 3 is a front view of the apparatus of FIGS. 1 and 2, in a super-extended configuration; and

FIG. 4 is a perspective view of part of the apparatus of FIGS. 1 to 3, from underneath.

### DETAILED DESCRIPTION

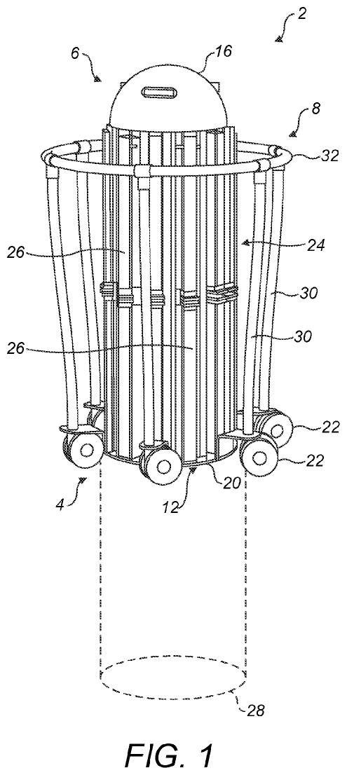

FIGS. 1 to 3 show the ultraviolet disinfecting apparatus 2 of a first embodiment of the invention. The apparatus 2 has a support structure 4 that defines an upright column 6, which in this case is generally cylindrical in shape, and an outer frame 8. The upright column 6 has a lower unit 10 which is attached to a base region 12, and an upper unit 14 which has a hemispherical sensor head assembly 16.

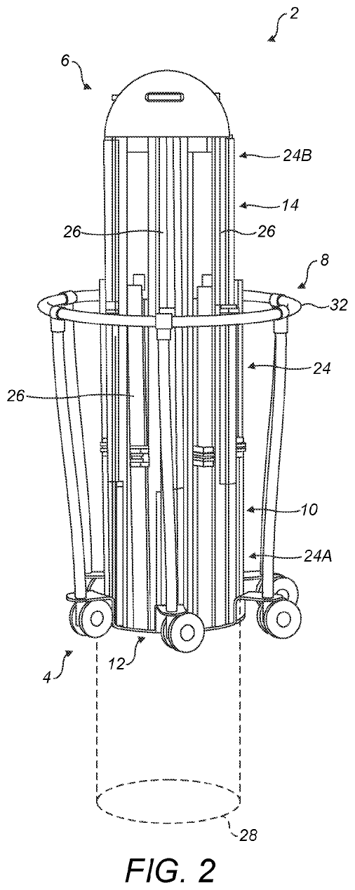

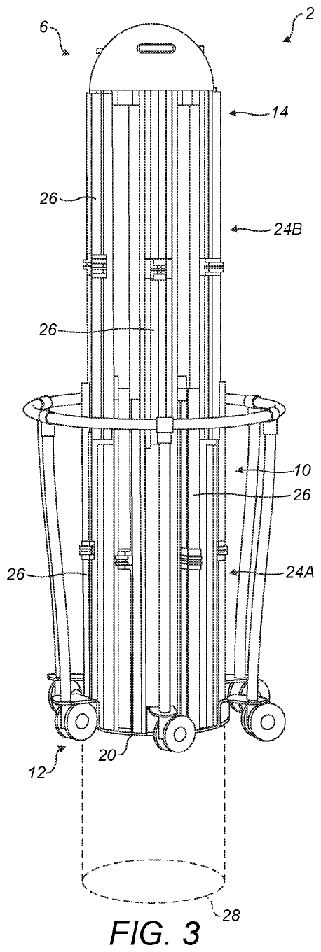

The two units 10, 14 interlock, the upper unit 14 being vertically extendible away from the lower unit 10 and the base region 12 as described in more detail later. FIG. 1 shows the apparatus 2 in a retracted configuration, FIG. 2 shows the apparatus 2 in an extended configuration, and FIG. 3 shows the apparatus in a super-extended configuration.

The apparatus 2 has a dual-voltage AC-DC adaptor (not visible) suitable for use at mains voltage of 110V and 230V. The apparatus 2 has a safety shut-off system as discussed later, but otherwise is controlled remotely.

The base region 12 of the lower unit 10 has a metal base plate 20, resting on six castor wheels 22. The base plate 20 supports a first set 24 of UV emitters. The first set 24 of UV emitters comprises a generally circular array of vertically-oriented fluorescent tube lamps, more specifically 95 W mercury vapour lamps 26 which, when energised, emit light at a wavelength of around 254 nm (i.e. UV-C light). Located radially inwards of each lamp 26 of the first set 24 is a corresponding ballast. The lamps 26 of the first set 24 of UV emitters are spaced apart from one another and positioned in a generally circular array which defines a perimeter 28 (which in this embodiment corresponds to the outer perimeter of the upright column 6).

When energised each of the lamps 26 emit UV light in a generally radially outward direction. In this case the first set 24 of emitters extends all the way around the perimeter and can therefore emit light in a radially outward direction through a complete 360 degree revolution around the perimeter 28 (and thus around the upright column 6).

The lamps 26 of first set 24 of UV emitters are divided into two sub-sets-a first sub-set 24A supported by the lower unit 10 of the support structure 4 and a second sub-set 24B supported by the upper unit 14. Since the lower and upper units 10, 14 are movable relative to one another, the sub-sets 24A, 24B are similarly movable. The lamps 26 of the two sub-sets 24A, 24B interlock with one another, with the circumferential spaces between the lamps 26 of the first subset acting as guide tubes for the interlocking lamps 26 of the second subset 24B. In the retracted position, shown in FIG. 1, the lamps 26 of the two sub-sets 24A, 24B overlap throughout their respective vertical heights. In the extended position shown in FIG. 2 the lamps 26 of the two sub-sets 24A, 24B overlap over around a third of their respective heights. In the super-extended position, shown in FIG. 3, the two sub-sets exhibit only a small degree of overlap with one another.

The lower unit 10 of the support structure 4 houses system electronics and a linear actuator (not visible) for raising the upper unit 14 of the apparatus, and provided on the outside of the lower unit 4 are one or more photodetectors (not visible) for monitoring UV light output in order to monitor the condition of the lamps 26.

The outer frame 8 runs around the upright column 6 and is attached to the lower unit 10. The outer frame 8 is made of metal, in this case aluminium, and has a set of legs 30 extending upwards from respective castor wheels 22, and a circular rim 32 supported by the legs and positioned at hand height so that an operator can push the apparatus 2 to move it around on the castor wheels 22.

The hemispherical sensor head assembly 16 positioned on top of the upper unit 14 houses an ultrasound distance sensor for measuring vertical height to the ceiling of a room. This sensor can be used to avoid the upper unit 14 being extended into the ceiling of a room, or the like. The sensor head assembly 16 also houses an optical distance-measuring module (not visible) for measuring the distance between the apparatus and features of a room (such as a wall, a pillar, a piece of furniture or the like). The distance-measuring module comprises an emitter and receiver and functions as a spatial sensor. It is rotatable through a horizontal plane and can tilt up to 90 degrees above and below its central horizontal position, so that the apparatus 2 can gather data on the room in which it is located. This data can be used to determine an optimal disinfection time in a manner which is not material to the present invention.

The apparatus of the first embodiment of the invention has an accompanying remote tablet controller (not shown). The tablet communicates bi-directionally with the disinfection apparatus via a Wi-Fi network and enables the operator to operate the apparatus in manual mode, or alternatively, run an automated cycle. Most of the control of the apparatus 2 is performed by the tablet computer, however the apparatus 2 also has a safety shut-off system which operates independently of the tablet controller. The safety shut-off system comprises a set of emergency stop buttons (not visible), and a set of passive infra-red sensors (not visible) for detecting movement in the vicinity of the apparatus 2, supported on the rim 32. A controller (not visible) inside the apparatus 2 immediately shuts off power to the first set 24 of UV emitters if one of the emergency stop buttons are pushed or if one of the passive infra-red sensors detects movement.

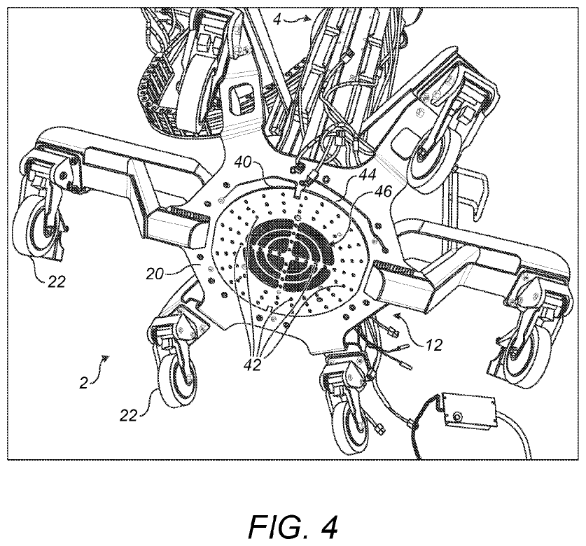

In addition to the first set 24 of UV emitters, the support structure 4 of the apparatus 2 supports a second set of UV emitters in the form of LED lamps. These are shown in FIG. 4, which shows the underside of the apparatus 2 with the first set of UV emitters and some of the support structure (for instance the outer frame) removed.

Referring now to FIG. 4 in combination with FIGS. 1 to 3, the second set 40 of UV emitters takes the form in the form of LED lamps 42 which, when energised, emit light at a wavelength of around 260 nm (i.e. UV-C light). The LED lamps 42 are positioned in a planar array, mounted to, and spread over a common thermally conductive backing 44. The backing 44, in turn, is attached to the underside of the base plate 20 of the base region 12 of the support structure 4. As such, the second set 40 of UV emitters is positioned on the base region 12.

With the LED lamps 42 mounted in this fashion, they are each positioned inside the perimeter 28 defined by the first set 24 of UV emitters. They are supported so as to be directed downwards, meaning that when energised they emit UV light in a downward direction. The first and second sets 24, 40 of UV emitters therefore emit UV light in different directions—the first set 24 emits UV light radially outwards, and the second set 40 emits UV light axially downwards. Accordingly, when the apparatus is placed in a room and is in use, UV light from the first set 24 of UV emitters is emitted outwards into the room and towards the walls of the room, while UV light from the second set 40 of UV emitters is emitted downwards into the region of the room which lies beneath the base region and inside the perimeter 28 (which region would otherwise be in a shadow of the light emitted by the first set 24).

The thermally conductive backing 44 of this embodiment defines a central vent 46, aligned with a corresponding aperture (not visible) in the base plate 20, through which air can be drawn into the apparatus 2 for cooling. In this case the backing 44 is of a sandwich construction, having uppermost and lowermost layers made of thermally conductive polymer, with a layer of metal electrical connecting pathways sandwiched therebetween. The LED lamps 42 are mounted on the lowermost layer and are all connected to a common driver circuit (not visible) through the metallic connection pathways.

Conventionally, LED lamps are mounted on or immediately adjacent to their driver circuits. However, this is not the case in the apparatus 2 of the present embodiment. Instead, the common driver circuit (not visible) is spaced apart from the LED lamps 42. More particularly, in this case the driver circuit is positioned within the upright column 6, and is connected to the backing 44 (and thus the LED lamps 42) via a wire which runs downward through an aperture in the base plate 20. With the driver circuit being positioned in the upright column, rather than being located on the underside of the base plate 20 along with the LED lamps 42, it is more protected from accidental knocks. Further, with the driver circuit not being accommodated beneath the base plate 20 it does not reduce the ground clearance of the apparatus 2.

In this embodiment, the thermally conductive backing 44 is attached to the metal base plate 20 in planar contact, and thus in thermal contact, therewith. This thermal contact allows heat generated by the LED lamps 42 to be conducted from the backing 44 and into the metal base plate 20. The heat can then travel from this metal structural component into other thermally conductive structural components such as the legs 30 of the outer frame 8, further dissipating it (and providing greater surface area for the heat to be lost into the air). The embodiment of the invention recognises that the use of LEDs on this base plate provides an especially compact arrangement because the structure itself can act as a heat sink (thereby removing the need for a separate bulky heat sink structure).

Whilst the present invention has been described and illustrated with reference to particular embodiments, it will be appreciated by those of ordinary skill in the art that the invention lends itself to many different variations not specifically illustrated herein. By way of example, in another embodiment of the invention the first set of UV emitters consists of a single helical mercury vapour lamp and the second set of UV emitters consists of a single LED lamp positioned in line with the axis of the helix of the mercury vapour lamp. In a further embodiment of the invention, which is a modification of the first embodiment, the first set of UV emitters comprises an array of mercury vapour fluorescent tubes arranged in the shape of a truncated cone, and the second set of UV emitters comprises an annular array of LED lamps positioned radially outward of the wider end of said cone, the LED lamps being angled so that they emit UV light obliquely and downward towards the axis of said cone. In another embodiment (not shown), the first set of emitter comprises a single fixed set, rather than the telescopic arrangement of the first embodiment.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. An ultraviolet disinfecting apparatus for disinfecting a room, the apparatus comprising:
a support structure with a base region;
a first set of UV emitters supported by the support structure to form a perimeter about which UV light can be emitted into said room; and
a second set of UV emitters supported by the support structure such that they can emit UV light into a region of said room which lies beneath the base region and inside the perimeter.

2. The ultraviolet disinfecting apparatus according to claim 1 wherein the UV emitters of the first set each take the form of a mercury vapour lamp, and the UV emitters of the second set each take the form of an LED lamp.

3. (canceled)

4. The ultraviolet disinfecting apparatus according to claim 2, wherein the support structure supports the first and second sets of UV emitters such that they can emit UV light in different directions.

5. The ultraviolet disinfecting apparatus according to claim 4 wherein the support structure comprises a base region, supports the first set of UV emitters such that they can emit UV light into said room, and supports the second set of UV emitters such that they can emit UV light into a region of the room which lies beneath the base region.

6. The ultraviolet disinfecting apparatus according to claim 5 wherein the first set of UV emitters are supported by the support structure to form a perimeter about which UV light can be emitted into said room, and the second set of UV emitters are supported by the support structure such that they can emit UV light into a region of the room which lies beneath the base region and inside the perimeter.

7. The ultraviolet disinfecting apparatus according to claim 6 wherein the first set of UV emitters extends all the way around the perimeter.

8. The ultraviolet disinfecting apparatus according to claim 7 wherein the second set of UV emitters is positioned inside the perimeter.

9. The ultraviolet disinfecting apparatus according to claim 8, wherein the second set of UV emitters is positioned on the base region of the support structure.

10. The ultraviolet disinfecting apparatus according to claim 9, wherein:
each of said LED lamps is provided on a thermally conductive backing;
the support structure comprises a thermally conductive structural component; and
the thermally conductive backing and the thermally conductive structural component are in thermal contact such that heat generated by the LED lamp is conducted into the thermally conductive structural component.

11. The ultraviolet disinfecting apparatus according to claim 10, wherein each of said LED lamps is electrically connected to an LED driver circuit which is spaced apart from said LED lamp.

12. The ultraviolet disinfecting apparatus according to claim 11, wherein the first set of UV emitters takes the form of an array of generally vertically-oriented fluorescent tube lamps.

13. The ultraviolet disinfecting apparatus according to claim 12, wherein:
the apparatus comprises a generally upright column;
the first set of UV emitters is supported by the support structure so that in use they emit UV light generally radially outwards from said column; and
the second set of UV emitters is supported by the support structure so that in use they emit UV light generally axially downwards from said column.

14. A room steriliser comprising a first ultraviolet illuminator arranged to emit UV light in a generally outward direction during use, and a second ultraviolet illuminator arranged to emit UV light in a generally downward direction during use.

15. The ultraviolet disinfecting apparatus according to claim 1, wherein the first set of UV emitters are configured to emit UV light towards the walls of said room, and a second set of UV emitters are arranged to emit UV light towards the floor of said room.

16. A floor-standing disinfecting apparatus for disinfecting a room, the apparatus comprising an upright section with an array of generally vertical UV-emitting tubes, and an array of UV-emitting LEDs positioned on a lower end face of the upright section.

17. The ultraviolet disinfecting apparatus according to claim 1, wherein:
the base comprises an upper side and a lower side;
the first set of UV emitters are upstanding from the upper side of the base and are configured to emit UV light into the room;
the second set of UV emitters comprise an array of LED lamps arranged on the lower side of the base and are configured to emit light into a region beneath the ultraviolet disinfecting apparatus during use.

18. The ultraviolet disinfecting apparatus according to claim 1 wherein the first set of UV emitters extends all the way around the perimeter.

19. The ultraviolet disinfecting apparatus according to claim 18, wherein the second set of UV emitters is positioned inside the perimeter.

20. The ultraviolet disinfecting apparatus according to claim 18, wherein the second set of UV emitters is positioned on the base region of the support structure.

21. The ultraviolet disinfecting apparatus according to claim 2, wherein:
each of said LED lamps is provided on a thermally conductive backing;
the support structure comprises a thermally conductive structural component; and
the thermally conductive backing and the thermally conductive structural component are in thermal contact such that heat generated by the LED lamp is conducted into the thermally conductive structural component.
